(19) United States
(12) Patent Application Publication
(10) Pub. No.: US 2022/0050094 A1
(43) Pub. Date: Feb. 17, 2022

(54) Application of the apparent volume of distribution in extraction technologies and pharmacokinetics

(71) Applicant: Michalakis Savva, Savannah, GA (US)

(72) Inventor: Michalakis Savva, Savannah, GA (US)

(21) Appl. No.: 17/163,730

(22) Filed: Feb. 1, 2021

Related U.S. Application Data
(63) Continuation-in-part of application No. 16/992,658, filed on Aug. 13, 2020.

(51) Int. Cl. G01N 33/15

(52) U.S. Cl. CPC G01N 33/15

(57) ABSTRACT

The present invention relates to the discovery of the apparent volume of distribution as an intensive physical property of matter and its use in the field of chemical separation sciences and pharmacokinetics. The invention provides various methods of determining the amount of substance in all phases, substance concentration in all phases, extraction efficiency, solvent capacity, phase volume needed to achieve a given extraction efficiency, the substance's apparent volumes of distribution with respect to each phase in the system and the partition coefficient or distribution ratio that defines the partitioning of the substance between two phases that are in contact. In the field of pharmacokinetic multiphasic compartment models the invention provides methods to determine the substance amount in the body from measured plasma substance concentration at all times in the one-compartment model and at times of momentary distribution equilibrium of the substance (t_{eq}) in multi-compartment models.

### CROSS REFERENCE TO RELATED APPLICATIONS

Ser. No. 16/992,658

### STATEMENT OF FEDERALLY SPONSORED RESEARCH

Not Applicable.

### NAMES OF THE PARTIES TO A JOINT RESEARCH AGREEMENT

Not Applicable.

### SEQUENCE LISTING

Not Applicable.

### BACKGROUND OF THE INVENTION

The literature is abundant with hundreds of different “definitions” for the volume of distribution e.g., a mathematical ratio, a fudge factor, a proportionality constant, an ideal, hypothetical, theoretical, imaginary or apparent volume that has no direct anatomical or physiological meaning but somehow it relates the drug concentration to the amount of drug in the body. In essence, the parameter was never understood, never defined scientifically and as a result it is not used in any scientific discipline other than pharmacokinetics which is also experiencing a decline in its use. Many of the simulations conducted by classical compartmental and physiological pharmacokinetic models during the last five years do not even report the value of the drug's apparent volume of distribution. Often, compartment volume is used synonymously to the apparent volume of distribution of a substance, terms like steady-state volume of distribution are used instead of apparent volume of distribution or volume of distribution is used instead of apparent volume of distribution.

The present invention relates to the discovery of the apparent volume of distribution (V_{d}) as a new property of matter and its application in the field of separation sciences and pharmacokinetics. I have characterized this new property using thought experiments of solute distribution in a biphasic system of constant volume, constant temperature and pressure (FIG. 1). The apparent volume of distribution is shown to be an intensive property of matter and it is now defined as the phase volume required to contain the total amount of solute added into the system while maintaining the solute phase concentration constant to its original value. Through these experiments I demonstrate that the apparent volume of distribution is related to the partition coefficient and the system phase volumes. It can be used in the field of solvent extraction in place of partition coefficient to optimize solvent extraction efficiency. In the field of pharmacokinetics, I demonstrate how to correctly calculate this property and how to use it to calculate drug amount in the body as a function of time and to assess the physiologically accessible by the drug compartment volumes.

### SUMMARY OF THE INVENTION

The apparent volume of distribution of a substance is defined for the first time from solute phase distribution experiments in closed systems as an intensive property of matter that its value is unaffected by the total mass of the substance and it is only affected by the partition coefficient and the phases volumes. There are as many substance apparent volumes of distribution as phases in a system. The present invention relates to the application of the newly discovered properties of the apparent volume of distribution in the field of solvent extraction and pharmacokinetics. New methods are described to determine substance apparent volume of distribution associated with each phase, solvent capacity and extraction efficiency of a system. In the field of pharmacokinetics, new methods are described that can calculate the drug amount in the body with time and the drug's apparent volume of distribution is the one-, multi-compartment and physiological pharmacokinetic models.

### BRIEF DESCRIPTION OF DRAWINGS

FIG. 1 Left: Solute added in a compartment containing only solvent 1 or solvent 2. Middle: Solute added in a biphasic system of immiscible solvents 1 and 2. Right: Solute partitions into the two phases and is allowed to escape out of the system from phase 1.

FIG. 2 Solute concentration (Left) and solute apparent volume of distribution (Right) in phase 1 (solid lines; C₁, V_{d,1}) and in phase 2 (dashed-lines; C₂, V_{d,2}). The mass of the solute in the system (FIG. 1, middle) was varied from 30 to 150 mg while the distribution coefficient and the volumes of the two phases were kept constant in all experiments, K_{2,1}=3, V₁=3 L, V₂=39 L (Vₛ=42 L).

FIG. 3 Phase solute concentration plotted as a function of total solute mass (xₛ) in the system. The maximum solvent capacity for solute extraction is the highest point on the equilibrium line (continuous line). The deviation from the equilibrium line observed at higher solute system amounts as indicated by the dashed-lines indicates system saturation. K_{2,1}=3, V₁=3 L, V₂=39 L.

FIG. 4 The amount of drug in the body after the first infusion (n=1), x_{s,1,eq}, was plotted against the drug concentration in the central compartment of the two-compartment mammillary model (FIG. 1, right) at momentary distribution equilibrium C_{1,1,eq}. Dose escalation studies were carried out at k₀ of 1, 1.5, 2, 2.5 and 3 mg/min 30-minute intermittent infusions. The sisomicin apparent volume of distribution was determined from the slope of the line to be 12.16 L (Savva, 2020, unpublished manuscript).

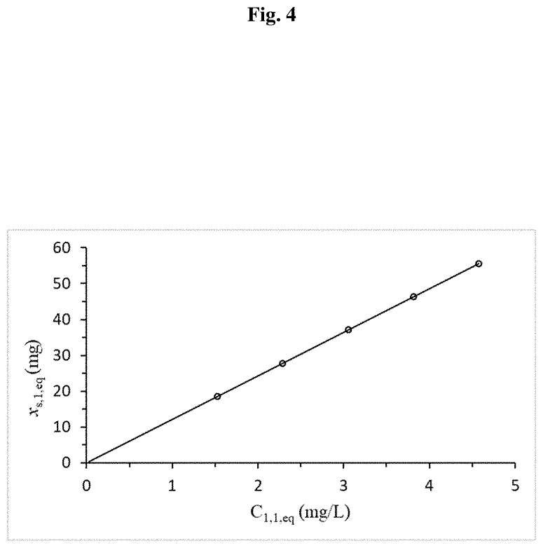

### DETAILED DESCRIPTION OF THE INVENTION

The work described in this invention was initiated as a thought experiment. It is not related to an accidental discovery of a substance's property after physical experimentation. Aiming to explain pharmacokinetic compartment models, I have looked for a property that stays constant regardless of the total mass of solute added in an isolated system composed of two immiscible solvents at constant volume, and relates the mass of the solute in the system to its concentration in one of the phases. A modified mass balance equation was used where its final state is expressed in terms of the drug concentration in one of the phases instead of the final concentration at equilibrium (eq. 1). I initially designated the solute's apparent volume of distribution as the phase volume that yields the total solute mass in the system when multiplied with the corresponding phase solute concentration (V_{d,1}·C₁=xₛ). I could have given this new property a new name but I didn't because this is how I believe the apparent volume of distribution of as substance should be defined. As I proceeded studying its properties in virtual distribution experiments, in closed and open systems, the above original definition was supplemented appropriately and became an official technical definition of the apparent volume distribution of a substance. The results of these thought experiments are so certain that it is totally unnecessary to physically perform them.

V₁·C₁+V₂·C₂=V_{d,1}·C₁ eq. 1

### The Approach

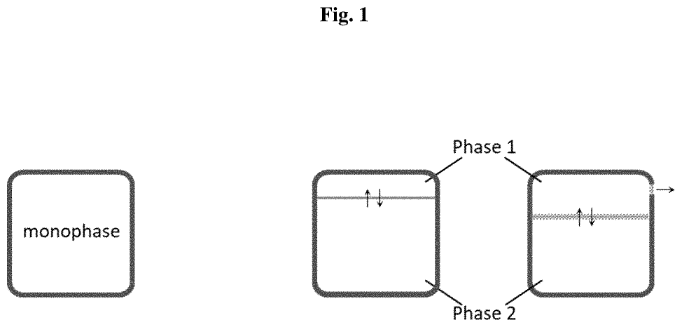

The apparent volume of distribution was initially studied in virtual distribution experiments of a solute in a closed biphasic system of constant volume (FIG. 1). The partition coefficient of the solute for the two phases, K_{2,1}, was set to be 3 times higher for phase 2 as compared to phase 1, in all experiments. At equilibrium, solute concentrations in the two phases, C₁ and C₂, were calculated using eq. 2 and 3.

$$\begin{matrix}{x}_{s} = {x}_{1} + {x}_{2} & eq . 2 \\ {K}_{2 , 1} = \frac{{x}_{2}}{{V}_{2}} \cdot \frac{{V}_{1}}{{x}_{1}} & eq . 3\end{matrix}$$

Where x₁, x₂, xₛ, V₁ and V₂ are the mass of solute in phase 1, solute mass in phase 2, the total solute mass in the two phases, the volume of phase 1 and the volume of phase 2. The volumes of the two phases make up the total volume of the system, Vₛ=V₁+V₂.

The volume of distribution of a substance that is added in a monophase made of pure solvent 1 or solvent 2 is the volume of that phase in the system (eq. 4; FIG. 1, left). The concept of the apparent volume of distribution does not apply in monophasic systems as there is nothing hypothetical about the volume of distribution of a substance in these systems.

$${V}_{d , 1} = {V}_{d , 2} = \frac{{x}_{s}}{{C}_{s}} = {V}_{s} \qquad eq . 4$$

On the other hand, the apparent volume of distribution of a solute associated with the solvents of a two-phase closed system (FIG. 1, middle panel) can be determined using a mass balance equation (eq. 5). As with the partition coefficient, there are as many volumes of distribution and as many apparent volumes of distribution as the number of phases in a system.

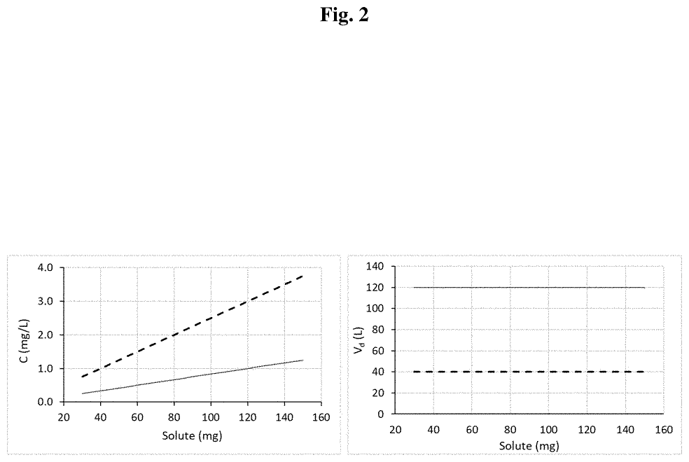

The results of the isothermic distribution experiments shown in FIG. 2 indicate that the apparent volume of distribution of a solute in a phase is independent of the mass of the solute in the system and it only depends on the distribution coefficient and the volumes of the two immiscible media that are in contact. We can use eqs. 5-10 to define the apparent volume of distribution associated with a solute in a phase as the volume of that phase that can form a solution with the total mass of the solute in the system at constant phase solute concentration. Adding for example, 60 mg of solute into a system composed of 3 L of phase 1 and 39 L of phase 2 with a partition coefficient K_{2,1}=3, results in V_{d,1}=120 L, V_{d,2}=40 L, C₁=0.5 mg/L, and C₂=1.5 mg/L. Accordingly, in order to accommodate 60 mg of the solute in phase 1 without causing any change in the original equilibrium solute concentration, you will have to add

$$\frac{{V}_{d , 1}}{{V}_{1}}$$

times bigger mass of solute (2400 mg) into a system composed of

$$\frac{{V}_{d , 1}}{{V}_{1}}$$

times larger phase volumes, that is, 120 L of phase 1 in contact with 1560 L of phase 2.

$$\begin{matrix}{V}_{d , 1} \cdot {C}_{1} = {V}_{1} \cdot {C}_{1} + {V}_{2} \cdot {C}_{2} & eq . 1 \\ {V}_{d , 1} = {V}_{1} + {V}_{2} \cdot {K}_{2 , 1} & eq . 5 \\ {V}_{d , 1} = \frac{{x}_{s}}{{C}_{1}} & eq . 6 \\ \frac{{V}_{1}}{{V}_{d , 1}} = \frac{{x}_{1}}{{x}_{s}} & eq . 7 \\ {V}_{d , 2} = \frac{{x}_{s}}{{C}_{2}} & eq . 8 \\ \frac{{V}_{d , 2}}{{V}_{2}} = \frac{{x}_{s}}{{x}_{2}} & eq . 9 \\ {K}_{2 , 1} = \frac{{C}_{2}}{{C}_{1}} = \frac{{V}_{d , 1}}{{V}_{d , 2}} & eq . 10\end{matrix}$$

It is important to say that the apparent volume of distribution can be determined using the distribution ratio instead of the distribution coefficient using the same equations described above. For example, if the weakly acidic compound, HA, dissociates in a pH-dependent manner in the aqueous phase (phase 1), eq. 5 can be written as,

$$\begin{matrix}{V}_{d , 1} = {V}_{1} + {V}_{2} \cdot {D}_{2 , 1} & eq . 11 \\ {V}_{d , 2} = \frac{{v}_{1}}{{D}_{2 , 1}} + {V}_{2} \\ Where , {D}_{2 , 1} = \frac{{[ H A ]}_{2}}{{[ {A}^{-} ]}_{1} + {[ H A ]}_{1}} . & eq . 12\end{matrix}$$

Eq. 10 can also be used to determine the distribution ratio.

$${D}_{2 , 1} = \frac{{c}_{2}}{{c}_{1}} = \frac{{[ H A ]}_{2}}{{[ {A}^{-} ]}_{1} + {[ H A ]}_{1}} = \frac{{v}_{d , 1}}{{v}_{d , 2}} \qquad eq . 13$$

It is worth noting that the increase in solute concentration in the two phases with increasing solute added into the system is linear and the inverse slope is equal to the apparent volume of distribution of the solute in each phase, in accord with equations 6 and 8. Also, the ratio of the two apparent volumes of distribution equals the value of the partition coefficient (FIG. 2, right, and eq. 10).

### Application of the Apparent Volume of Distribution in Extraction Technologies and Pharmaceutical Dosage Forms

1. The efficiency of an extraction method is evaluated by the fraction of the solute that moves into the extracting phase (eq. 7). Using the concept of the apparent volume of distribution the extraction efficiency can be calculated from a single concentration measurement and the known volume of the assayed phase using eq. 6 and eq. 7 (Example 1).
2. The capacity of a solvent to extract a substance can be evaluated by addition of increasing amounts of substance in the system. The apparent volume of distribution can be calculated from the slope of linear regression analysis of measured equilibrium phase solute concentrations as a function of solute mass in the system (FIG. 2, left) and can be used to optimize the product load in a solvent system in order to achieve a certain concentration of the substance in the extracting phase.

### Application of the Apparent Volume of Distribution in Pharmacokinetics

In the classical pharmacokinetic compartmental modeling, compartments that receive the drug with a different rate but are otherwise compositionally similar have the same apparent volume of distribution. Each compartment has its own solute's apparent volume of distribution only if the compartments constitute different phases. The ratio of the drug concentration in two phases at equilibrium equals the value of the partition coefficient regardless of the solute total mass and volume of the two phases in the system. As with the partition coefficient, there are as many solute apparent volumes of distribution as the number of phases in a system but unlike the partition coefficient, the apparent volume of distribution of a substance is dependent on the actual phase volumes that are in contact. The ratio of the solute concentration maybe dictated exclusively by the value of the partition coefficient but the actual solute concentration in the two phases is also controlled by the actual phase volumes. As I have already demonstrated, at constant phase volumes, phase solute concentrations are directly proportional to the total mass of the solute in the system. This is why the apparent volume of distribution of a drug is such an important pharmacokinetic property.

In vivo, the apparent volume of distribution of a drug can be defined as the volume of the assayed phase that can contain a drug dose while maintaining the drug concentration in the assayed phase constant. Drug concentrations are usually measured in the plasma, but the apparent volume of distribution of a drug can be related to the whole phase or compartment that includes extracellular fluid other than the plasma. Therefore, changes in drug dose are reflected in changes of drug concentration in the assayed phase (eq. 6). It is on this basis that drug doses can be correlated with measurements of drug concentration in body fluids to accurately assess the apparent volume of distribution of a drug for the assayed phase and the actual volume of that phase.

In general, there are three conditions necessary for determining a substance apparent volume of distribution in multicompartment models and/or multi-phasic systems:
1. Constant total system volume
2. Constant phase volumes
3. Solute mass transfer equilibrium between different phases or between single phase kinetically different compartments

### The One-Compartment Model

The one-compartment pharmacokinetic model is an open model that assumes instantaneous distribution of the drug from the central circulation to all other tissues in the body. The above statement is so important that we must rephrase it: the one-compartment open model assumes instantaneous solute distribution/redistribution equilibrium between the vascular fluid and all other tissues at all times. As we have already shown, the apparent volume of distribution of a substance in a system composed of a single compartment phase must be equal to the compartment volume (eq. 6). Then why the apparent volume of distribution of most lipophilic drugs that follow a one-compartment model is larger than the volume of the total body fluid? There can be only one answer for that: contrary to current beliefs the one-compartment model doesn't have to be a single phase. Although it lumps all tissues with similar rates of drug uptake in a single compartment, it is wrong to assume that the compartment is uniform. In fact, the one-compartment model recognizes that plasma drug concentration is some multiple of the drug concentration in other tissues and it acknowledges the presence of other phases via the changes in the pharmacokinetic parameters that are calculated from concentration measured in the “central” compartment. It is not, however, designed to see any of those phases that are maybe in contact with the “central” compartment. So, although it calculates correctly the V_{d} of a drug from its initial plasma drug concentration measured at time zero after an IV bolus dose, it fails to gather information about the actual volume of the assayed phase except in few distinct cases. The apparent volume of distribution of a drug in a one-compartment model can be determined from its initial plasma drug concentration at time zero, because in an open system the elimination process of the drug starts as soon as the drug is injected into the central circulation and thus time zero is the only time that the whole dose is present in the body. Drugs with an apparent volume of distribution about 3 L must follow the one-compartment model. Apparently, these molecules do not have any affinity for any tissue other than the plasma or their size is prohibitively large to escape into the extravascular environment via the capillaries. Drugs with a 3 L<V_{d}≤15 L are hydrophilic drugs that may diffuse through the capillary endothelium but are otherwise unable to diffuse inside cells. These drugs do not have to follow a one-compartment model if their distribution into the various extracellular fluids is kinetically different but an apparent volume of distribution in that range suggests the presence of only a single phase in all drug accessible compartments. Drugs with an apparent volume of distribution about 40 L or 60% of total body weight in healthy adults, have access to the total body fluid. Using eq. 5, an average V_{extracellular}=15 L and V_{intracellular}=27 L, we find that the only way to get a drug apparent volume of distribution equal to the total volume of body fluids is when the drug partitions in extracellular and intracellular fluids with an equal affinity (K_{2,1}=1).

Lipophilic drugs are known to have apparent volumes of distribution much higher than the total body fluid leaving little drug in the plasma. Some of them can be simulated using a one-compartment pharmacokinetic model. Apparently, distribution of the drug to extracellular fluids and other tissues is taking place at the same rate, and hence, drug disposition can be modeled using a single bi-phasic compartment. The second phase is the tissues that have higher affinity for the drug. In agreement to our theory, the product of the tissue-plasma partition coefficient and the tissue phase volume explains why the V_{d,a} of these drugs can be much higher than total body fluid volume (eq. 14). Therefore, even in the absence of any information about the high drug affinity tissues, eq. 14 can still be used to calculate the drug's V_{d,a} from measured plasma drug concentration.

$${C}_{a} = \frac{{x}_{s}}{( {V}_{a} + {V}_{b} \cdot {K}_{b , a} )} \qquad eq . 14$$

Where a and b are the two phases present in the single compartment and V_{d,a}=Vₐ+V_{b}·K_{b,a}.

In the open one-compartment pharmacokinetic model there is a continuous substance distribution equilibrium between phases at all times. This statement has such profound consequences now that we have defined the apparent volume of distribution. Once the V_{d} of a drug is determined, eq. 6 or eq. 14 can be used to calculate the amount of drug in the body at all times because the open one-compartment pharmacokinetic model assumes a state of permanent substance distribution/redistribution equilibrium between the vascular, extravascular and intracellular fluids. We don't need to know the elimination rate constant nor the clearance to calculate an intravenous dose aimed to achieve a certain plasma drug concentration or to calculate the amount of drug in the body from measured drug plasma concentrations.

Eq. 14 (or eq. 6) is another version of the clinically useful loading dose equation (eq. 15) that can be used to calculate loading or booster doses in order to quickly raise the plasma drug concentration to the desired levels within therapeutic range in the plasma, strictly in the one-compartment open model.

D_{L}=C·V_{d} eq. 15

C and V_{d} are the plasma drug concentration at time zero and the drug's apparent volume of distribution associated with the plasma or the plasma-associated phase in the open one-compartment model. As we will see, this equation cannot be used in multi-compartment models as in those models, solute distribution equilibrium is not instantaneous and therefore it is not the dose that is related to the plasma drug concentration but the amount of drug remaining in the body at the time where the momentary distribution equilibrium is observed.

The only exception to this rule is when the drug is administered by a slow constant rate continuous intravenous infusion. In this case, at steady state there is essentially no fluctuation of the drug concentration in the plasma and regardless of compartment model, eq. 15 can used to calculate loading or booster doses to achieve desired steady state plasma or central phase drug concentrations. Calculating the apparent volume of distribution at steady state conditions during constant rate continuous IV drug infusion can be done using eq. 16.

$${V}_{d , 1} = \frac{{k}_{0} \cdot {t}_{1 / 2}}{{C}_{1 , ave , ss}} \qquad eq . 16$$

The rate of drug administration k₀ is in units of mass per time, t_{1/2} is the systemic half-life of the drug and C_{1,ave,ss}=Cₐᵥₑ=Cₛₛ is the steady state plasma drug concentration upon constant rate continuous intravenous infusion of the drug.

### Multi-compartment Models

The classical pharmacokinetic compartment models were never explained because the apparent volume of distribution was never understood. The apparent volume of distribution is an intensive property of matter that cannot be observed or measured directly but it can be calculated from measurements of the volumes and substance concentration in two phases that are in contact.

It is generally believed that the compartment volumes of multi-compartment pharmacokinetic models bear no physiological significance. As we have seen, the apparent volume of distribution is equal to the amount of drug in the system over the drug concentration in the plasma. Since in multi-compartment mammillary pharmacokinetic models the plasma drug concentration is equal to the amount of drug in the central compartment over the volume of the central compartment, the drug apparent volume of distribution associated with the central compartment must be related to the actual volume of the central compartment (eq. 7). Therefore, the compartment volume assessed by regression analysis methods of measured plasma drug concentration with time must be the physiologically drug-accessible volume of the central compartment.

In the simplest of multicompartment pharmacokinetic models, the two-compartment model, the volume of the central compartment V₁ can be determined by eq. 17. As distribution of drug in the peripheral compartment is not instantaneous, at zero time all the drug is in the central compartment and the extrapolated to time zero plasma drug concentration (C_{1,0}) must be related to the real volume of the central compartment.

$${V}_{1} = \frac{D o s {e}_{IV}}{{C}_{1 , 0}} = \frac{D o s {e}_{IV}}{A + B} \qquad eq . 17$$

The accurate assessment of the volume of the central compartment V₁ from experimental measurements of drug concentration using regression techniques is extremely important not only because it is the actual physiological volume of the central compartment but because it is also used to calculate V_{d,1} after computing the volume of the peripheral compartment and the drug concentration in the peripheral compartment as a function of time. One of the problems of the model are the conditions of distributional equilibrium at which V₂ is currently being determined.

$$\frac{{k}_{21}}{{k}_{12}} = \frac{{x}_{1}}{{x}_{2}} = \frac{{V}_{1}}{{V}_{2}} \qquad eq . 18$$

Eq. 18 indicates that V₂ can be calculated from V₁ and the intercompartmental drug transfer rate constants or from the mass of the solute in the two compartments when solute distribution rates in the two compartments are equal. The left-hand side of eq. 18 derived from eq. 19, is stating that at equilibrium there is an equal mass transfer rate of solute between the two compartments in the system, that is,

k₁₂·x₁−k₂₁·x₂=0 eq. 19

The caveat of the method is that drug distributional equilibrium is decided from Fick's law based on conditions of equal drug concentration in the two compartments. Under these conditions the ratio of the intercompartmental transfer rate constant equals the volume ratio of the two phases (eq. 18, right-hand side). In other words, the method is accurate only for drugs that have an approximate tissue-plasma partition coefficient K_{2,1}=1 with the kinetically different compartments of the model being compositionally very similar.

In our recent article, we have calculated V_{d,1} of sisomicin after multiple intermittent intravenous infusions in a two-compartment model using eq. 6 (Savva, 2020, unpublished manuscript). Drug concentrations in central and peripheral compartments were calculated with equations described in U.S. patent application Ser. No. 16/992,658. As the concept of the steady-state plasma drug concentration is unrelated to the apparent volume of distribution, the apparent volume of distribution of a drug can be assessed after administration of single or multiple doses from drug concentrations that are measured exactly at the time at which the distribution rates of drug into the two compartments are equal (momentary distribution equilibrium). We must recognize that in the more realistic open two-compartment model (FIG. 1, right) distributional equilibrium is not a lasting one. This is true for all routes of drug administration except for the constant rate continuous intravenous infusion where intercompartmental drug distribution reaches a long-lasting equilibrium when the plasma drug concentration reaches steady state conditions. The time of momentary distributional equilibrium (t_{eq}) was determined as a function of time and dose number from eq. 19. It was estimated that solute distribution between the two compartments reaches equilibrium 29.5-to-22.7 minutes after infusion has stopped and 1.8-to-4.6 minutes after the drug amount or concentration has reached its peak in the peripheral compartment, depending on the infusion number. Keep in mind that with repetitive drug administration the xₛ on eq. 6, being the amount of drug in the system and not the drug dose, is not a constant quantity. The drug was administered every 180 minutes with a short infusion of 30 minutes. The value of the V_{d,1} is calculated from the amount of drug in the body and the concentration in the central compartment (C_{1,eq}) at t_{eq}, as a function of dose number, to be equal to 12.16 L (Table I).

TABLE I
The solute's apparent volume of distribution associated with the central
compartment was calculated from the solute mass in the central and peripheral
compartment at t_{eq} after repetitive intermittent intravenous infusions in a
mammillary two-compartment model (Savva, 2020, unpublished manuscript).

| n | x_{1,eq} (mg) | x_{2,eq} (mg) | C_{1,eq} (mg/L) | V_{d,1} (L) |
|---|---|---|---|---|
| 1 | 16.71 | 20.39 | 3.05 | 12.16 |
| 2 | 23.37 | 28.51 | 4.27 | 12.16 |
| 3 | 25.82 | 31.50 | 4.72 | 12.16 |
| 4 | 26.71 | 32.59 | 4.88 | 12.16 |
| 5 | 27.03 | 32.98 | 4.94 | 12.16 |
| 6 | 27.15 | 33.12 | 4.96 | 12.16 |

The volumes of the central and peripheral compartments as calculated by the original authors of the work were 5.17 L and 6.61 L (Pechere et al., 1976). We claim that these volumes represent the physiological volumes of extracellular fluid compartments that receive the drug with different kinetic rates. Total exclusion of the drug from intracellular fluids is most plausible considering the highly polar and hydrophilic nature of sisomicin. Alternatively, the peripheral compartment could be considered to be interstitial fluid as a different phase of slightly higher drug affinity, where steric hindrance and binding to the negatively charged components of the extracellular matrix retards drug movement. In any case, our analysis suggests that classical pharmacokinetic modeling is able to provide an accurate description of the drug concentration with time for hydrophilic drugs that have access only to intravascular and interstitial fluids. The explicit solutions to the two-compartment model as a function of real time provide estimates of drug concentration upon repeated dosing (U.S. patent application Ser. No. 16/992,658). These drug concentrations at momentary distribution equilibrium (t_{eq}) can be used to determine the drug's apparent volume of distribution.

Ideally, solute transfer at the interface between two immiscible phases reaches steady state when C₂=K_{2,1}·C₁. We can combine this thermodynamic relationship denoting equal chemical potential of solute in each phase with the steady state eq. 19 where the mass transfer rate of the solute between the two phases does not change with time.

$$\frac{{k}_{12}}{{k}_{21}} = \frac{{x}_{2}}{{x}_{1}} = \frac{{C}_{2}}{{C}_{1}} \cdot \frac{{V}_{2}}{{V}_{1}} = {K}_{2 , 1} \cdot \frac{{V}_{2}}{{V}_{1}} \qquad eq . 20$$

Eq. 20 contains two unknowns, the partition coefficient, K_{2,1} and V₂. In order to calculate the volume of peripheral compartment(s) the “tissue-plasma” partition coefficient(s) must first be determined. If the peripheral compartment is a single organ, then the apparent volume of distribution V_{d,1} determined from a measured plasma drug concentration and the amount of drug in the body at t_{eq} using eq. 24, can be used to compute the organ-plasma partition coefficient from V₁ and the corresponding calculated organ volume using eq. 5. Nonetheless, an apparent volume of distribution greater than the total body water is a definite indication of the presence of another phase in the system.

Combining eq. 5 with eq. 20 yields a familiar equation,

$${V}_{d , 1} = {V}_{1} \cdot ( 1 + \frac{{k}_{12}}{{k}_{21}} ) \qquad eq . 21$$

Eq. 21, can be used to assess a substance apparent volume of distribution associated with the phase of the central compartment, but only if the central compartment is a single phase. Using eq. 21, V_{d,1} was calculated to be equal to 11.78 L. The equilibrium time of intercompartmental drug distribution t_{eq} is calculated from eq. 19 whereas the compartment volumes were estimated by regression analysis of measured plasma drug concentration with time, hence the small difference between the two calculated values of the apparent volume of distribution (Table I).

Our extensive literature search for lipophilic drug multi-compartment pharmacokinetic model data simulation indicated that the central compartment of lipophilic drugs is consistently reported having a volume, much higher than the extracellular fluid volume and frequently higher than total body water. It is possible that for these drugs the central compartment of a multi-compartment mammillary model contains two kinetically indistinguishable phases instead of a single phase. The plasma is part of a phase a that is in contact with phase b in the central compartment and therefore measured plasma drug concentrations are affected by the drug distribution in phase b within the central compartment. The apparent volume of distribution of the drug in phase a is equal to the volume of phase a and the sums of the products of the volume of phase b and phase 2 with the corresponding partition coefficients (eq. 22) for a two-compartment or a multi-compartment mamimmary model (eq. 23).

V_{d,1a}=V₁ₐ+V_{1b}·K_{b,a}+V₂·K_{2,1a} eq. 22

V_{d,1a}=V₁ₐ+V_{1b}·K_{b,a}+Σᵢ₌₂^{∞}Vᵢ·K_{i,1a} eq. 23

Without the tissue-plasma partition coefficient, it will not be possible to determine neither the actual volume of the central compartment from the plasma drug concentration at time zero nor that of the peripheral compartment. However, measured plasma drug concentrations do reflect the drug that is present in both phases of the central compartment and thus, the drug's apparent volume of distribution can still be determined with eq. 6. It is important to remember that the total amount of drug in the body and the plasma drug concentration must be determined at times of momentary distribution equilibrium, t_{eq} (eq. 24).

$${V}_{d , 1 , a} = \frac{{x}_{s , eq}}{{C}_{1 , a , eq}} \qquad eq . 24$$

In physiologically-based pharmacokinetic modeling (PBPK) predictions of the apparent volume of distribution are carried out using the familiar equation below:

Vₛₛ=Vₚ+Σᵢ₌₁ⁿV_{t,i}·K_{t,i,p} eq. 25

The Vₛₛ, Vₚ, Vₜ and K_{t,p} are the apparent volume of distribution, the volume of plasma, the volume of tissue and the tissue-plasma partition coefficient, respectively. Firstly, the term steady state (apparent) volume of distribution must be replaced with V_{d,1}. The conditions that are necessary to determine a drug's apparent volume of distribution in multi-compartment models are equal mass transfer rates between compartments and not steady-state plasma drug concentration. The only mode of administration that ensures a near zero fluctuation in steady state plasma drug concentration and hence long lasting equal intercompartmental mass transfer rates, is the constant rate continuous intravenous infusion. In PBPK modeling of V_{d,1} every tissue compartment constitutes a different phase in contact with the central circulation but the partition coefficient is multiplied by the tissue volume instead of the tissue phase volume. The accuracy of the model could be improved using the volume of central compartment instead of the plasma volume and by adjusting the tissue volume without the estimated interstitial fluid volume that is exchanged with the central phase.

As we previously demonstrated in examples 1-4, one of the advantages of the apparent volume of distribution is that it can be used to determine the partition coefficient in any multiphasic system e.g., suspensions, emulsions, solid lipid nanoparticles, liposomes, adherent or suspension cells, tissue homogenates or whole tissue, without having to measure the drug concentration in both phases. In a series of in vitro equilibrium titration experiments, drug is added into the closed system (xₛ) and the V_{d,1} is determined from a measured drug concentration in the bulk media (C₁) using

$${V}_{d , 1} = \frac{{x}_{s}}{{C}_{1}} .$$

The partition coefficient can be subsequently determined from the estimated tissue volume (Vₜ) using eq. 5,

$${K}_{t , 1} = \frac{{V}_{d , 1} - {V}_{1}}{{V}_{t}} ,$$

without having to measure the intracellular drug concentration. The drug's in vitro apparent volume of distribution with respect to the tissue is

$${V}_{d , t} = \frac{{V}_{d , 1}}{{K}_{t , 1}}$$

and the in vitro phase drug concentration is

$${C}_{t} = \frac{{x}_{s}}{{V}_{d , t}} .$$

The experimentally determined values of K_{t,1,p} can be used in PBPK modeling to provide a more accurate simulation of in vivo drug concentration in the tissues using the arterial drug concentration and the tissue blood flow.

### EXAMPLE 1

50 mL of 0.10 M aqueous drug solution (phase 1) is to be shaken with 10 mL of organic solvent (phase 2). The organic solvent-water distribution coefficient K_{2,1} of the drug is 6.5. (a) What is the drug concentration remaining in the aqueous phase at equilibrium? (b) What is the extraction efficiency? (c) What is the extraction efficiency after two extractions? (d) How many extractions do we need to perform to extract greater than 99.9% of the solute in the organic phase (e) What volume of the organic solvent must be used to extract exactly 99.9% of the solute?

### Solution

$${K}_{2 , 1} = \frac{{C}_{2}}{{C}_{1}} = 6.5 , {V}_{2} = 10 mL , {V}_{1} = 50 mL \qquad ( a )$$

### Using eq. 5,

V_{d,1}=V₁+V₂·K_{2,1}=50+10·6.5=115 mL

The amount of drug in the system is:

$${x}_{s} = {V}_{1} \cdot {C}_{1} = 0.05 L \cdot 0.10 M = 0.005 mol$$

$${C}_{1} = \frac{{x}_{s}}{{V}_{d , 1}} = \frac{0.005}{0.115} = 0.0 \; 4348 M$$

(b) The extraction efficiency

$$\frac{{x}_{1}}{{x}_{s}}$$

of the aqueous phase can be calculated using eq. 7:

$$\frac{{x}_{1}}{{x}_{s}} = \frac{{V}_{1}}{{V}_{d , 1}} = \frac{50}{115} = 0.4348$$

Thus, the extraction efficiency of the organic solvent is 1−0.43=0.57
(c) After two extractions,

$${( \frac{{x}_{1}}{{x}_{s}} )}_{2} = {( \frac{{V}_{1}}{{V}_{d , 1}} )}^{2} = 0.19$$

Thus, the extraction efficiency of the organic solvent is 1−0.19=0.81 or 81%
(d) To achieve extraction efficiency of 99.9%,

$${( \frac{{x}_{1}}{{x}_{s}} )}_{n} = {( \frac{{V}_{1}}{{V}_{d , 1}} )}^{n} = 0.001 \\ n = \frac{ln ( 0.00 \; 1 )}{ln ( \frac{{V}_{1}}{{V}_{d , 1}} )} = 8.29$$

Thus, to achieve extraction efficiency >99.9% we need to carry out 9 extractions.
(e) To achieve extraction efficiency

$$\frac{{x}_{2}}{{x}_{s}}$$

of 99.9% we are going to have to use much more organic solvent. It is important to remember that there will be a new apparent volume of distribution for a new phase volume and a new total system volume. Thus, we need to calculate the new V_{d,1}.

$$\frac{{x}_{1}}{{x}_{s}} = 0.0 \; 01 = \frac{{V}_{1}}{{V}_{d , 1}} = \frac{50}{{V}_{d , 1}}$$

$${V}_{d , 1} = 50 L$$

$$Using eq . 10 , \\ {V}_{d , 2} = \frac{{V}_{d , 1}}{{K}_{2 , 1}} = \frac{50 , 000}{6.5} = 7 , 692.31 mL$$

$$\frac{{x}_{2}}{{x}_{s}} = 0.999 = \frac{{V}_{2}}{{V}_{d , 2}} = \frac{{V}_{2}}{7 \; 692.31} \\ {V}_{2} = 7 , 684.6 mL$$

Alternatively, using eq. 5,

$${V}_{2} = \frac{{V}_{d , 1} - {V}_{1}}{{K}_{2 , 1}} = 7 , 684.6 mL$$

### EXAMPLE 2

At constant phase volumes (constant V_{d,1} and V_{d,2}) one can use the equilibrium line of the distribution isotherm in FIG. 2 or eq. 6 to determine the amount of solute required to be added in order to obtain the extractant at a certain concentration in one of the phases and vice versa.

$${V}_{d , 2} = \frac{{x}_{s}}{{C}_{2}} \qquad eq . 6$$

Consider for example an extraction system composed of V₂=39 L, V₁=3 L,

$${K}_{2 , 1} = \frac{{C}_{2}}{{C}_{1}} = 3 ,$$

V_{d,2}=40 L. If you wish to achieve an extractant concentration in phase 2 equal to 2 mg/L you are going to have to add in the system,

xₛ=V_{d,2}·C₂=40 L·2 mg/L=80 mg

### EXAMPLE 3

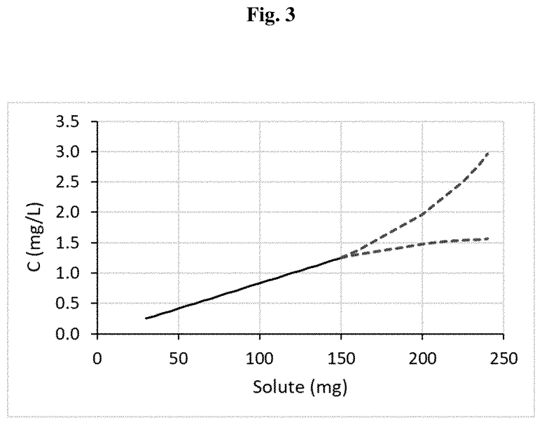

At constant phase volumes (constant V_{d,1} and V_{d,2}) one can carry out isothermic distribution experiments to determine the extraction capacity of the solvent system as the point at which a deviation of linearity in the xₛ-C plot is observed (FIG. 3).

### EXAMPLE 4

Determine the partition coefficient from V_{d,1} or V_{d,2} (at constant phase volumes). This method is extremely useful when the substance concentration is difficult or cannot be measured in one of the phases e.g., intracellular concentration in suspension cells, adherent cells, cell homogenate, tissue homogenate, whole tissue, emulsions, lipid solid nanoparticles, liposomes or any other two- or multiple-phase delivery system.

### Solution

The bulk phase can be separated from the dispersed one by centrifugation, ultrafiltration, gel permeation or any other method. The apparent volume of distribution can be calculated from the total mass of substance added in the system and one concentration measurement in the bulk phase using eq. 6.

$${V}_{d , 1} = \frac{{x}_{s}}{{C}_{1}} \qquad eq . 6$$

The distribution coefficient can be calculated from the apparent volume of distribution and the actual volumes of the two phases using eq. 5.

$${K}_{2 , 1} = \frac{{V}_{d , 1} - {V}_{1}}{{V}_{2}}$$

The apparent volume of distribution of the substance and the substance concentration in the second phase are,

$${V}_{d , 2} = \frac{{V}_{d , 1}}{{K}_{2 , 1}} and {C}_{2} = \frac{{x}_{s}}{{V}_{d , 2}} ,$$

respectively.

### EXAMPLE 5

Determine the tissue-plasma partition coefficient from V_{d,1} or V_{d,2} in ex situ tissue perfusion studies.

### Solution

Drug solution simulating plasma is administered by IV bolus or via an infusion pump into afferent blood vessels of the isolated tissue. Drug concentration is measured in both afferent and efferent blood vessels of the isolated tissue. At equilibrium (steady-state if the drug is supplied by a constant rate continuous infusion) the drug's apparent volume of distribution can be calculated from the mass of drug in the tissue (or dose) and the drug concentration in the efferent blood vessels using eq. 24 (or eq. 6).

$${V}_{d , 1} = \frac{{x}_{s , eq}}{{C}_{1 , e q}}$$

Repeating the process for different doses should yield a xₛ-C plot similar to that of FIG. 2. The inverse slope of the equilibrium line is equal to V_{d,1}. The tissue-plasma partition coefficient K_{b,a} can be determined from eq. 14 where Vₐ and V_{b} are the constant volume of perfusate which is recycled via the pump and the volume of intracellular tissue fluid, respectively.

### DEFINITION OF TERMS

x: drug amount.
x₁: mass of solute in phase 1 or compartment 1.
xₛ: total solute mass in the system.
V₂: volume of distribution of phase 2 or just volume of phase 2.
Vₛ: the total volume of a system.
V_{d}: substance's apparent volume of distribution.
V_{d,1}: substance's apparent volume of distribution with respect to solvent 1.
Phase: A distinct and homogeneous state of a system.
K_{2,1}: Partition or distribution coefficient of a substance is defined as the ratio of the concentration of a substance in a single definite form in the extract (phase 2) to its concentration in the other phase, in the same form, at equilibrium

$$( {K}_{2 , 1} = \frac{{C}_{2}}{{C}_{1}} ) .$$

D_{2,1}: Partition or distribution ratio of a substance is defined as the ratio of the total analytical concentration of a substance in the extract (phase 2) regardless of its chemical form to its total analytical concentration in the other phase. For example, the distribution ratio of a weak acid, HA, between water and organic phases is

$${D}_{2 , 1} = \frac{{K}_{2 , 1} \cdot [ {H}^{+} ]}{[ {H}^{+} ] + {K}_{a}} .$$

Another example could be the liquid-liquid extraction of a metal-ligand complex ([MLn]). In this case the distribution ratio,

$${D}_{2 , 1} = \frac{{[ MLn ]}_{2}}{[ {M}^{n +} ] + {[ MLn ]}_{1}} .$$

Solvent extraction: The process of transferring a solute from a solid matrix, gas or liquid phase to another immiscible or partially miscible liquid phase in contact with it.
Extractant: An immiscible or partially miscible phase made of pure solvent or a liquid solution that is used to extract the substance from another phase that is in contact.
τ: dosing interval in units of time.
T: infusion time.
k: first-order elimination rate constant.
k₀: zero-order rate of drug infusion which is also the maintenance dose (D_{M}) in IIV.
D_{M}: drug maintenance dose.
D_{L}: drug loading or booster dose.

### REFERENCES

Pechere J C, Pechere M M, Dugal R. Clinical pharmacokinetics of sisomicin: two-compartment model analysis of serum data after IV and IM administration. Eur J Clin Pharmacol. 1976; 10: 251-256.
Savva, M (2020) Real-time analytical solutions as series formulas and Heaviside functions for multiple intermittent intravenous infusions in the one- and two-compartment pharmacokinetic models. Submitted for publication.

As the inventor of the apparent volume of distributaion and the pharmacokinetic multiphasic compartment models, I claim:

1. The use of the apparent volume of distribution (V_{d}) of a substance in any process, where the substance of interest partitions in and between two or more phases, that are in solid, liquid or gaseous state, they are in contact and are immiscible or partially miscible, for the purpose of determining:
the amount of substance in all phases;
the substance concentration in all phases;
the extraction efficiency;
the solvent capacity;
the solubility of the substance;
the phase volume needed to achieve a given extraction efficiency;
the substance's apparent volumes of distribution with respect to each phase in the system;
the partition coefficient or distribution ratio that defines the partitioning of the substance between the two phases that are in contact;
the substance amount in the body from measured plasma substance concentration at all times using eq. 6 or eq. 14 in the one-compartment model;
the substance amount in the body from measured plasma substance concentration using eq. 24 in multi-compartment models at times of momentary distribution equilibrium of the substance between the different compartments that are in contact (t_{eq});
the substance's apparent volume of distribution from multiple dose-escalation studies from the slope of the equilibrium line of the plot of plasma drug concentration against amount of drug in the body in the one- and multiple compartment models;
the substance's apparent volume of distribution after constant rate continuous intravenous infusion using eq. 16
$$( {V}_{d , 1} = \frac{{k}_{0} \cdot {t}_{1 / 2}}{{C}_{1 , ave , ss}} ) ,$$
regardless of compartment model;
the substance's apparent volume of distribution in multicompartment models using eq. 21
$$( {V}_{d , 1} = {V}_{1} \cdot ( 1 + \frac{{k}_{12}}{{k}_{21}} ) )$$
if the central phase is a single phase or if the V_{d,1} as determined by another method was found to be less than 0.6-to-0.8 L/kg; and
the value of the apparent volume of distribution from eq. 23 (V_{d,1a}=V₁ₐ+V_{1b}·K_{b,a}+Σᵢ₌₂^{∞}Vᵢ·K_{i,1a}) where V₁ is the volume of the central compartment and not plasma and Vᵢ is the volume of tissue phase which is different that the central phase or the volume of the tissue without the interstitial fluid that is exchanged with the central phase.

2. The exclusive use of the apparent volume of distribution in multiphasic compartment models composed of kinetically different compartments with respect to the rate of drug uptake/release, where each compartment can be composed of one or more phases and each phase can have the same or different drug affinity.

3. That compartment volumes as determined by regression analysis of experimental data in classical pharmacokinetic compartment models, where each compartment may be composed of one or more phases, represent real physiologically relevant body fluid volumes accessible by a substance.
